(19) United States
(12) Patent Application Publication
(10) Pub. No.: US 2025/0070172 A1
(43) Pub. Date: Feb. 27, 2025

(54) ANTHRAQUINONE-BASED ACTIVE MATERIAL

(71) Applicants: MITSUBISHI HEAVY INDUSTRIES, LTD., Tokyo (JP); NAGOYA DENKI EDUCATIONAL FOUNDATION, Aichi (JP)

(72) Inventors: Koichiro Hirayama, Tokyo (JP); Toshiyasu Kiyabu, Tokyo (JP); Yasushi Morita, Aichi (JP); Tsuyoshi Murata, Aichi (JP); Aya Ito, Aichi (JP); Shigemitsu Okada, Aichi (JP)

(73) Assignees: MITSUBISHI HEAVY INDUSTRIES, LTD., Tokyo (JP); NAGOYA DENKI EDUCATIONAL FOUNDATION, Aichi (JP)

(21) Appl. No.: 18/721,302

(22) PCT Filed: Dec. 19, 2022
(86) PCT No.: PCT/JP2022/046562

§ 371 Date: Jun. 18, 2024

(30) Foreign Application Priority Data
Dec. 22, 2021 (JP) 2021-208037

(51) Int. Cl. H01M 4/60; C07C 51/09; H01M 8/18

(52) U.S. Cl. CPC H01M 4/60; C07C 51/09; H01M 8/18

(57) ABSTRACT

An anthraquinone-based active material for a redox flow battery includes a first compound represented by the following chemical formula:

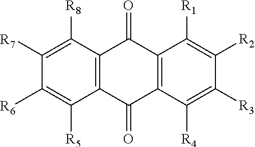

where at least one of the R₁ to R₈ is a hydroxy group, and at least one of the R₁ to R₈ is an alkoxy group.

### TECHNICAL FIELD

The present disclosure relates to an anthraquinone-based active material for a redox flow battery.

The present application claims priority based on Japanese Patent Application No. 2021-208037 filed on Dec. 22, 2021, the entire content of which is incorporated herein by reference.

### BACKGROUND ART

Redox flow batteries are suitable for storing a large amount of electricity because the electricity storage amount can be freely designed according to the tank capacity, and they are expected to be applied to leveling the demand for electricity, including natural energy. Redox flow batteries are characterized in that they are composed of a cell that charges and discharges and an electrolyte tank for power storage, and a pump is used to circulate electrolyte for charging and discharging.

Currently, redox flow batteries that use vanadium as an active material in electrolyte are the mainstream, but due to the recent surge in vanadium prices and other factors, redox flow batteries that use organic materials or metal complexes as the active material are being developed. For example, Patent Document 1 describes a redox flow battery that uses anthraquinone or naphthoquinone as a negative electrode active material, and a number of anthraquinones with sulfo groups are illustrated. Patent Document 2 describes, although not an active material itself, a redox flow battery that uses as the active material a composition containing a coordination compound with a redox non-innocent ligand coordinated to a metal center, and as the redox non-innocent ligand, a number of anthraquinones with various functional groups bonded to the 1- to 8-positions of anthraquinones are illustrated. Non-Patent Documents 1 and 2 also describe compounds with various functional groups or elements bonded to the 1- to 8-positions of anthraquinones.

### CITATION LIST

### Patent Literature

Patent Document 1: JP6574382B

Patent Document 2: JP2019-514170A (translation of a PCT application)

### Non-Patent Literature

Non-Patent Document 1: K. Lin, Q. Chen, M. R. Gerhardt, L. Tong, S. B. Kim, L. Eisenach, A. W. Valle, D. Hardee, R, G. Gordon, M, J. Aziz, M. P. Marshak, Science, 349 (2015) 1529-1532 Non-Patent Document 2: D. G. Kwabi, K. Lin, Y. Ji. F. Kerr, M. Goulet, D. D. Porcellinis, D. P. Tabor, D. A. Pollack, A. Aspuru-Guzik, R. G. Gordon, M. J. Aziz, Joule 2, 19 (2018) 1894-1906

### SUMMARY

### Problems to be Solved

Although any functional group or element can be bonded to the 1- to 8-positions of

anthraquinones, certain combinations of them may cause practical problems. For example, Non-Patent Document 2 describes the problems of 2,6-dihydroxyanthraquinone (2,6-DHAQ), which has a significant drop in capacity during charge and discharge, as well as 2,6-bis(3′-carboxypropyloxy)-9,10-anthraquinone (2,6-DBEAQ), which has less drop in capacity during discharge but has a low cell voltage.

In view of the above, an object of at least one embodiment of the present invention is to provide an anthraquinone-based active material having a good balance between the cell voltage of a redox flow battery and the suppression of capacity decrease during discharge.

### Solution to the Problems

In order to achieve the above object, an anthraquinone-based active material according to the present disclosure for a redox flow battery includes a first compound represented by the following chemical formula:

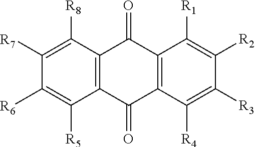

where at least one of the R₁ to R₈ is a hydroxy group, and at least one of the R₁ to R₈ is an alkoxy group.

### Advantageous Effects

With the anthraquinone-based active material of the present disclosure, the presence of hydroxy group ensures an appropriate cell voltage of the redox flow battery, and the presence of alkoxy group suppresses the capacity decrease during discharge of the redox flow battery, thus achieving a good balance between the cell voltage and the suppression of capacity decrease during discharge.

### BRIEF DESCRIPTION OF DRAWINGS

FIG. 1 is a graph showing an experiment result of Example 1.

### DETAILED DESCRIPTION

The following is a description of the anthraquinone-based active material (hereinafter, simply “active material” unless it is specifically necessary to add “anthraquinone-based”) according to embodiments of the present disclosure. The embodiment to be described below indicates one aspect of the present disclosure, does not intend to limit the disclosure, and can optionally be modified within a range of a technical idea of the present disclosure.

### <Basic Structure of Active Material of Present Disclosure>

The active material of the present disclosure is an active material that dissolves in electrolyte on the negative electrode side of the redox flow battery in a discharged state, and includes a compound represented by the following chemical formula (1). When this compound is used as the negative electrode active material of the redox flow battery, through a redox reaction, the compound is converted to a reduced form in which the oxygen atoms double-bonded to the 9- and 10-positions of the anthraquinone skeleton are converted to hydroxy groups, or vice versa. Specifically, when the redox flow battery performs a discharge operation, an oxidation reaction occurs in which the reduced form is converted to the compound, and when the redox flow battery performs a charge operation, a reduction reaction occurs in which the compound is converted to the reduced form.

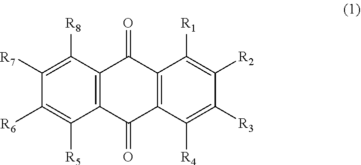

In chemical formula (1), at least one of R₁ to R₈ bonded to the anthraquinone skeleton is a hydroxy group, and at least one of R₁ to R₈ is an alkoxy group (—OR). In the alkoxy group, R bonded to the oxygen atom has 1 to 6 carbon atoms, and when R has 4 to 6 carbon atoms, it has a linear or branched structure. The bond between carbon atoms constituting R is not limited to a single bond, but may include a double or triple bond. R may contain an ether bond. Further, at least one of the carbon atoms constituting R may be bonded to a halogen or any functional group, such as a hydroxy group, sulfo group, amino group, nitro group, carboxy group, phosphoryl group, thiol group, or alkyl ester, instead of hydrogen.

When the compound with such a structure is used as the negative electrode active material of the redox flow battery, the presence of hydroxy group in the active material ensures an appropriate cell voltage of the redox flow battery, and the presence of alkoxy group suppresses the capacity decrease during discharge of the redox flow battery, thus achieving a good balance between the cell voltage and the suppression of capacity decrease during discharge.

### <Variation of Active Material of Present Disclosure>

In the basic structure of the active material of the present disclosure, one or more hydroxy groups and alkoxy groups are required among R₁ to R₈, and the number of them is not limited, but a compound with a structure that limits the number of hydroxy groups to one may be used as the negative electrode active material of the redox flow battery. The active material with such a structure is simpler than active materials having multiple hydroxy groups, and the synthesis method and procurement of raw materials are easier. In addition, in the case of alkaline electrolyte, a hydroxy group of an active material functions as an acid, which neutralizes alkali in the electrolyte. This requires addition of extra alkali to the electrolyte for the neutralization. In this context, the above-described active material is more advantageous than active materials having two or more hydroxy groups in that less hydroxy groups in the active material requires less alkali to be added to the electrolyte.

Further, a compound with a structure that limits the number of hydroxy groups and alkoxy groups to one each may be used as the negative electrode active material of the redox flow battery. The active material with such a structure is simpler than active materials having multiple hydroxy groups and multiple alkoxy groups, and the synthesis method and procurement of raw materials are easier.

In the compound with a structure that limits the number of hydroxy groups and alkoxy groups to one each, a structure with a hydroxy group bonded to the 2-position and an alkoxy group bonded to the 6-position is preferred. The raw material for synthesizing this compound is 2,6-dihydroxyanthraquinone (2,6-DHAQ), which is industrially mass-produced and is easy to procure. The compound with this structure can be synthesized from 2,6-DHAQ by reaction with an organic alkylating agent (RX) in the presence of a base or acid, as shown in the following chemical reaction formula (2). In chemical reaction formula (2), R is any alkyl group and X is any desorbing group such as halogen, tosylate, mesylate, sulfonate, phosphonate, or 1-imino-2-(trichloro) ethyloxy. As the base, NaH, NaOH, KOH, or K₂CO₃ can be used, as well as alkoxide, triethylamine, diisopropylethylamine, or diazabicycloundecene.

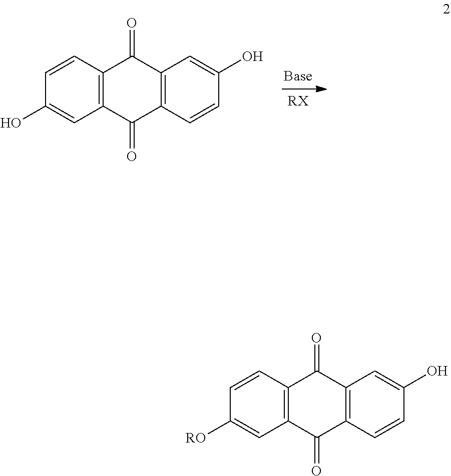

The alkoxy group bonded to the 6-position may be O(CH₂)ₙCOOH having a carboxyl group (n is a natural number from 1 to 6). The active material having a carboxyl group improves its solubility in electrolyte on the negative electrode side.

A compound with a structure that limits the number of hydroxy groups to two but not the number of alkoxy groups (provided that at least one alkoxy group is contained) may be used as the negative electrode active material of the redox flow battery. In the active material with such a structure, the electron-donating properties of the two hydroxy groups combine to further increase the electron density of the anthraquinone skeleton and shift the redox potential to lower values, thereby increasing the cell voltage of the redox flow battery.

Further, a compound with a structure that limits the number of hydroxy groups and alkoxy groups to two each may be used as the negative electrode active material of the redox flow battery. The active material with such a structure is simpler than active materials having three or more hydroxy groups and/or three or more alkoxy groups, and the synthesis method and procurement of raw materials are easier.

In the compound with a structure that limits the number of hydroxy groups and alkoxy groups to two each, a structure in which two of R₂, R₃, R₆, and R₇ are hydroxy groups and the remaining two are alkoxy groups is preferred. The raw material for synthesizing this compound is 2,3,6,7-tetrahydroxyanthraquinone (2,3,6,7-THAQ), which can be produced at high yield and is easy to procure. The compound with this structure can be synthesized from 2,3,6,7-THAQ by reaction with an organic alkylating agent (RX) in the presence of a base, as shown in the following chemical reaction formula (3).

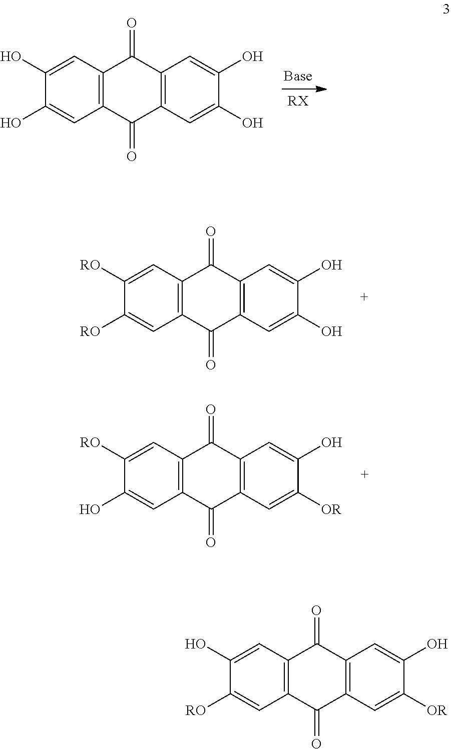

In the compound with a structure in which two of R₂, R₃, R₆, and R₇ are hydroxy groups and the remaining two are alkoxy groups, the two alkoxy groups may be each O(CH₂)ₙCOOH (n is a natural number from 1 to 6). The compound with this structure can have the following three structures of compounds (4) to (6). The active material having a carboxyl group improves its solubility in electrolyte on the negative electrode side. For example, n=3 shows a suitable solubility of 0.6 M/1M-KOH in alkaline electrolyte.

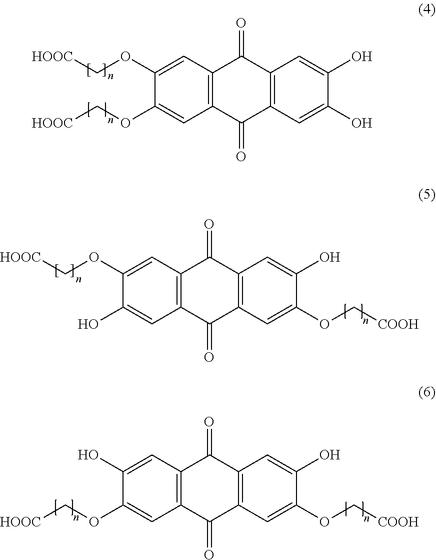

The active material is not limited to only the compound having the above structure (first compound), but may be a mixture of the first compound and a second compound represented by the following chemical formula (7).

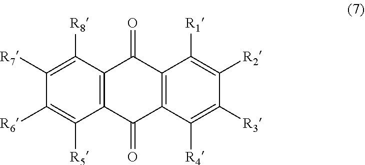

In chemical formula (7), the second compound is a compound in which at least one of R₁′ to R₈′ is a hydroxy group and the remainder of R₁′ to R₈′ is a hydrogen atom, or a compound in which at least one of R₁′ to R₈′ in the chemical formula is an alkoxy group and the remainder of R₁′ to R₈′ is a hydrogen atom, or a mixture thereof. Three examples of the mixture of the first compound and the second compound are illustrated in the following chemical formulas (8) to (10).

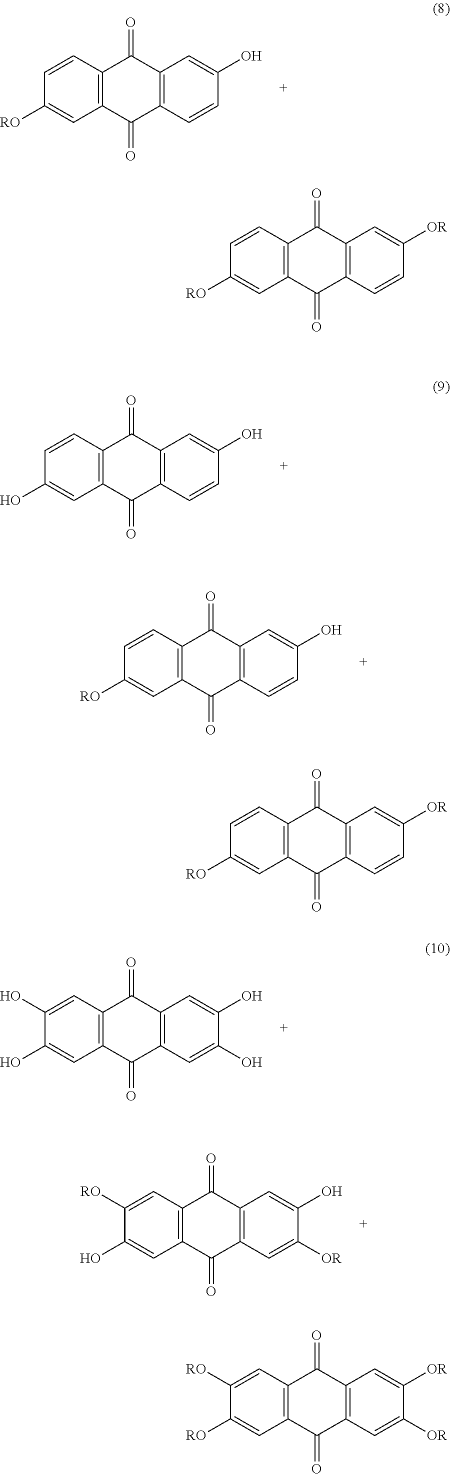

When using the mixture of the first and second compounds of the above structure as the active material, the cell voltage and electrolyte viscosity can be adjusted by adjusting the mixing ratio of anthraquinones with different properties.

In order to obtain the above effect, the ratio of the mass of the first compound to the mass of the active material is preferably 0.3 or more, more preferably 0.4 or more, more preferably 0.5 or more, more preferably 0.6 or more, more preferably 0.7 or more, more preferably 0.8 or more, more preferably 0.9 or more, more preferably 0.95 or more, most preferably 0.99 or more.

As illustrated by the following chemical formula (11), when the molecule of the first compound is inverted with respect to center C of the central six-membered ring of anthraquinone skeleton, a combination of R₁ to R₈ preferably has a different structure from that before the inversion. In other words, the first compound is preferably an anthraquinone-based substance without i-symmetry. Since the molecule of the first compound with this structure has dipole moments, polarization occurs in the molecule, improving the solubility in polar electrolyte and increasing the capacity density of the redox flow battery.

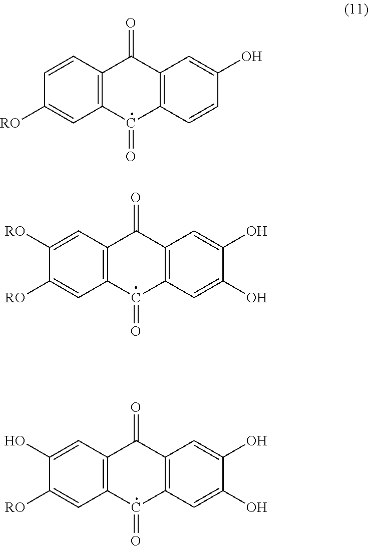

If a compound containing a sulfo group is dissolved in neutral to alkaline electrolyte and used as the active material, the redox potential of the active material may increase, the cell voltage may decrease, and the voltage efficiency may decrease. Therefore, the compound represented by chemical formula (1) preferably does not contain a sulfo group. However, if the compound represented by chemical formula (1) has a hydroxy group, the redox potential tends to decrease due to its effect, so compounds in which the number of sulfo groups is less than the number of hydroxy groups can be included in the compound represented by chemical formula (1).

### EXAMPLES

In Example 1, where potassium ferrocyanide trihydrate and potassium ferricyanide were used as the positive electrode active material and (2-(3′-carboxypropyloxy)-6-hydroxy-9,10-anthraquinone (2,6-MHMBEAQ) was used as the negative electrode active material, the cell voltage and the rate of capacity decrease during discharge in the redox flow battery were measured.

The positive electrode electrolyte was prepared by dissolving 5.76 g (13.6 mmol) potassium ferrocyanide trihydrate and 1.80 g (5.45 mmol) potassium ferricyanide in 1.0 mol/L potassium hydroxide aqueous solution and diluting in a measuring cylinder to 68.2 mL. The negative electrode electrolyte was prepared by dissolving 0.816 g (2.5 mmol) of 2,6-MHMBEAQ in 1 mol/L potassium hydroxide aqueous solution and diluting in a measuring cylinder to 25 mL.

2,6-MHMBEAQ was synthesized by the procedure represented by the following chemical reaction formula (12). The synthesis is outlined as follows: from 2,6-DHAQ as the starting material, an intermediate with an alkoxy group in which hydrogen of one hydroxy group is replaced by ethyl butanoate is synthesized, and from this intermediate, the target substance, 2,6-MHMBEAQ, is synthesized.

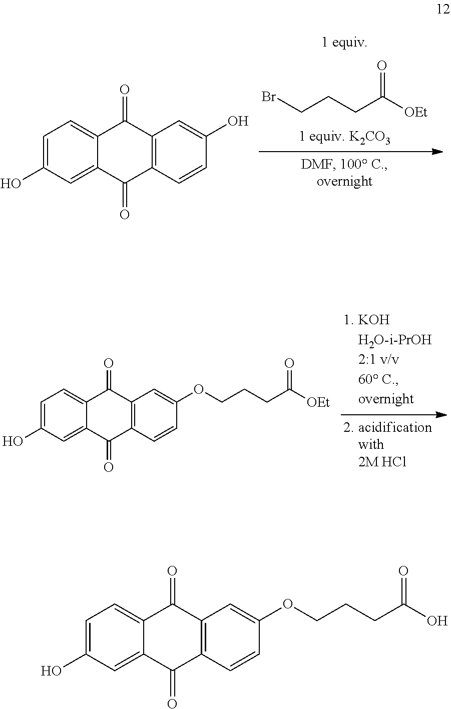 [SMILES: O=C1c2ccc(O)cc2C(=O)c2ccc(O)cc21 ; CCOC(=O)CCCBr ; CCOC(=O)CCCOc1ccc2c(c1)C(=O)c1ccc(O)cc1C2=O ; O=C(O)CCCOc1ccc2c(c1)C(=O)c1ccc(O)cc1C2=O]

In a 1 L eggplant flask, 40.0 g (167 mmol) of 2,6-DHAQ (Tokyo Chemical Industry Co., Ltd.) and 500 mL of N,N-dimethylformamide (DMF) were added, and 23.1 g (167 mmol) of potassium carbonate was added with stirring, followed by addition of 23.9 mL (167 mmol) of ethyl 4-bromobutanoate. The temperature rise was then started, and the mixture was stirred at 100° C. for 17 hours. After standing to cool, 600 mL of distilled water was added, the precipitate was suction filtered, and the residue was washed with distilled water. To the filtrate (pH>9), 6 M hydrochloric acid was added with stirring. After hydrochloric acid was added until the pH of the filtrate was less than 3 and carbon dioxide was no longer produced by addition of hydrochloric acid, the mixture was stirred at room temperature for 1 hour. The precipitate was transferred to a 200 mL centrifuge tube and centrifuged to separate the sediment. The sediment was suction filtered and washed with distilled water, followed by vacuum drying at 80° C. for 6 hours to obtain 11.4 g of a mixture of raw material and intermediate. The resulting solid was ground to a powder and suspended in 200 mL of chloroform. Insoluble material was removed by suction filtration, and washing was performed with 200 ml of chloroform until soluble material was completely dissolved. Through this operation, 11.1 g of unreacted raw material was recovered. The filtrate was again suction filtered to completely remove insoluble material, and the filtrate was vacuum concentrated. The residue was suspended in distilled water, suction filtered, washed, and vacuum dried at 80° C. for 4 hours to obtain 6.96 g of intermediate as a reddish brown solid (12% yield).

Next, 6.96 g (19.6 mmol) of the intermediate was put in a 1L eggplant flask with 190 mL of isopropyl alcohol and 380 mL of distilled water. Then, 4.48 g (79.9 mmol) of potassium hydroxide was added, the temperature rise was started, and the mixture was stirred at 60° C. for 20 hours. After standing to cool, 550 mL of distilled water was added, the mixture was transferred to a 2L erlenmeyer flask, and 2 M hydrochloric acid was added with stirring until the pH was less than pH. After stirring for 2 hours, the sediment was separated by centrifugation. The supernatant and sediment were separately suction filtered, and the residues were washed with distilled water. The residues were vacuum dried at 80° C. for 4 hours to obtain 6.25 g of the target substance (98% yield from the intermediate).

The redox flow battery used in measurements was made by the inventors of this disclosure themselves. This redox flow battery has a configuration in which the positive electrode cell and the negative electrode cell are separated from each other by an ion exchange membrane (Nafion (registered trademark), NR-212). Each cell has a 21 mm×21 mm meandering channel as the flow path for electrolyte. Each cell has a porous electrode (20 mm×20 mm) made of carbon paper.

Each electrolyte was contained in a Schlenk flask and bubbled with inert gas (nitrogen) for at least 5 minutes to remove dissolved oxygen. Each Schlenk flask was kept at 30° C. using an aluminum block thermostatic bath (ALB-121, Scinics Corporation). A pump (smooth flow pump QI-100-VF-P-S, Takunami Corporation) was used to flow each electrolyte at 65 mL/min through the flow path of each cell and circulate it between each cell and each Schlenk flask.

A charging/discharging device (ACD-01, Aska Electronic Co., Ltd.) was electrically connected to a current collector (carbon separator made by the inventors of this disclosure using conductive carbon resin) in each cell with a cable, and a charge-discharge cycle was conducted with a current value of 400 mA (current density 100 mA/cm²) by repeating constant-current charging with the upper cutoff voltage limit at 1.4 V and constant-current discharge with the lower cutoff voltage limit at 0.6 V.

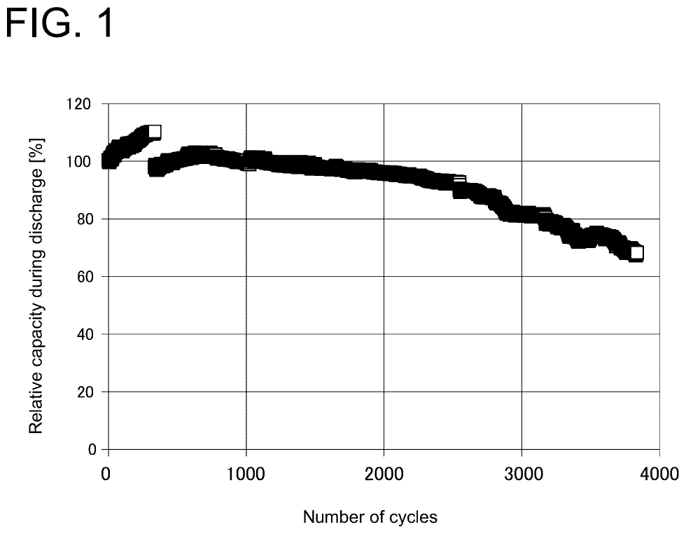

During the cycle, the voltage (cell voltage) between the positive and negative electrodes during discharge was measured, and the discharge capacity was measured for each cycle. The measurement result for each cycle is shown in FIG. 1. The relative capacity on the vertical axis of FIG. 1 is the ratio of the discharge capacity of each cycle to the discharge power of the second cycle. The rate of capacity decrease was calculated from the percentage change between the value of capacity in the second cycle and the value of capacity in the 3834th cycle, using the following calculation expression, n=3834.

$$Rate of capacity decrease = ( 1 - ( Dn / D 2 ) 1 / t ) \times 100$$

Dn: Relative capacity in nth cycle
D2: Relative capacity in second cycle (D2=1)
n: Number of cycles
t: Number of days elapsed

The aforementioned Patent Document 2 describes the cell voltage of redox flow batteries with potassium ferrocyanide as the positive electrode active material or 2,6-DHAQ and 2,6-DBEAQ as the negative electrode active material (respectively, Comparative Examples 1 and 2 here) in Table S2, and the rate of capacity decrease during discharge in Figure S7. Table 1 below shows the cell voltage and the rate of capacity decrease during discharge for Example 1 and Comparative Examples 1 and 2.

TABLE 1

| | | | Rate of capacity |
|---|---|---|---|
| | | Cell voltage | decrease during |
| | Negative electrode active material | (V) | discharge (%/day) |
| Example 1 | 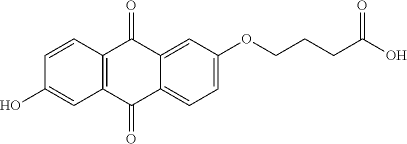 [SMILES: O=C(O)CCCOc1ccc2c(c1)C(=O)c1ccc(O)cc1C2=O] | 1.1 | 0.5 |
| | 2,6-MHMBEAQ | | |
| Comparative Example 1 | 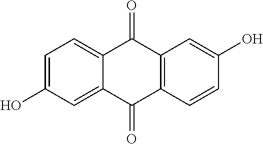 [SMILES: O=C1c2ccc(O)cc2C(=O)c2ccc(O)cc21] | 1.20 | ~5 |
| | 2,6-DHAQ | | |
| Comparative Example 2 | 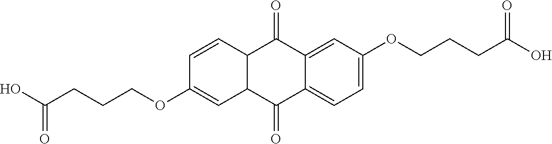 [SMILES: O=C(O)CCCOC1=CC2C(=O)c3ccc(OCCCC(=O)O)cc3C(=O)C2C=C1] | 1.05 | 0.01 |
| | 2,6-DBEAQ | | |

Comparing Comparative Example 1 with Comparative Example 2, the former has a higher cell voltage than the latter, but a higher rate of capacity decrease during discharge. In other words, having only hydroxy groups results in a higher cell voltage but a higher rate of capacity decrease during discharge, while having only alkoxy groups results in a lower cell voltage but a lower rate of capacity decrease during discharge. In contrast, having one hydroxy group and one alkoxy group, as in Example 1, results in values of cell voltage and rate of capacity decrease during discharge between Comparative Example 1 and Comparative Example 2. It can be thus said that Example 1 has a better balance of cell voltage and rate of capacity decrease during discharge than Comparative Examples 1 and 2, and that the cell voltage of the redox flow battery is appropriate and the rate of capacity decrease during discharge of the redox flow battery is suppressed.

The contents described in the above embodiments would be understood as follows, for instance.
[1] An anthraquinone-based active material according to one aspect is an anthraquinone-based active material for a redox flow battery, including a first compound represented by the following chemical formula:

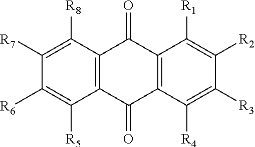

where at least one of the R₁ to R₈ is a hydroxy group, and at least one of the R₁ to R₈ is an alkoxy group.

With the anthraquinone-based active material of the present disclosure, the presence of hydroxy group ensures an appropriate cell voltage of the redox flow battery, and the presence of alkoxy group suppresses the capacity decrease during discharge of the redox flow battery, thus achieving a good balance between the cell voltage and the suppression of capacity decrease during discharge.
[2] An anthraquinone-based active material according to another aspect is the anthraquinone-based active material of [1], in which only one of the R₁ to R₈ is a hydroxy group.

With this configuration, the active material has a simpler structure than active materials having multiple hydroxy groups, and the synthesis method and procurement of raw materials are easier.
[3] An anthraquinone-based active material according to still another aspect is the anthraquinone-based active material of [2], in which only one of the R₁ to R₈ is an alkoxy group.

With this configuration, the active material has a simpler structure than active materials having multiple hydroxy groups and multiple alkoxy groups, and the synthesis method and procurement of raw materials are easier.
[4] An anthraquinone-based active material according to still another aspect is the anthraquinone-based active material of [1], in which only two of the R₁ to R₈ are hydroxy groups.

With this configuration, the electron-donating properties of the two hydroxy groups combine to further increase the electron density of the anthraquinone skeleton and shift the redox potential to lower values, thereby increasing the cell voltage of the redox flow battery.
[5] An anthraquinone-based active material according to still another aspect is the anthraquinone-based active material of [4], in which only two of the R₁ to R₈ are alkoxy groups.

With this configuration, the active material has a simpler structure than active materials having three or more hydroxy groups and/or three or more alkoxy groups, and the synthesis method and procurement of raw materials are easier.
[6] An anthraquinone-based active material according to still another aspect is the anthraquinone-based active material of [5], in which two of the R₂, R₃, R₆, and R₇ are hydroxy groups and remaining two of the R₂, R₃, R₆, and R₇ are alkoxy groups.

With this configuration, the raw material for synthesizing this active material is 2,3,6,7-tetrahydroxyanthraquinone, which can be produced at high yield and is easy to procure.
[7] An anthraquinone-based active material according to still another aspect is the anthraquinone-based active material of any of [1] to [6], in which the R₂ is a hydroxy group, and the R₆ is an alkoxy group.

With this configuration, the raw material for synthesizing this active material is 2,6-dihydroxyanthraquinone, which is industrially mass-produced and is easy to procure.
[8] An anthraquinone-based active material according to still another aspect is the anthraquinone-based active material of any of [1] to [7], in which the alkoxy group is O(CH₂)ₙCOOH, where n is a natural number from 1 to 6.

With this configuration, the presence of carboxyl group improves the solubility of the active material in electrolyte.
[9] An anthraquinone-based active material according to still another aspect is the anthraquinone-based active material of any of [1] to [8], in which when the molecule of the first compound is inverted with respect to center of a central six-membered ring of anthraquinone skeleton, a combination of R₁ to R₈ has a different structure from that before the inversion.

With this configuration, since the molecule of the first compound has dipole moments, polarization occurs in the molecule, improving the solubility in polar electrolyte and increasing the capacity density of the redox flow battery.
[10] An anthraquinone-based active material according to still another aspect is the anthraquinone-based active material of any of [1] to [9], in which the first compound does not contain a sulfo group.

If the first compound contains a sulfo group, the use of the first compound dissolved in neutral to alkaline electrolyte as the active material may increase the redox potential of the active material, decrease the cell voltage, and decrease the voltage efficiency. In contrast, if the first compound does not contain a sulfo group, such inconvenience can be avoided.
[11] An anthraquinone-based active material according to still another aspect is the anthraquinone-based active material of any of [1] to [9], in which the first compound contains a sulfo group, and the number of sulfo groups is less than the number of hydroxy groups.

If the first compound has a hydroxy group, the redox potential tends to decrease due to its effect, so the inconvenience due to the active material having a sulfo group can be avoided as long as the number of sulfo groups is less than the number of hydroxy groups.
[12] An anthraquinone-based active material according to still another aspect is the anthraquinone-based active material of any of [1] to [11], further including a second compound represented by the following chemical formula:

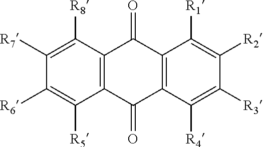

The second compound is a compound in which at least one of the R₁′ to R₈′ is a hydroxy group and remainder of the R₁′ to R₈′ is a hydrogen atom, or a compound in which at least one of the R1′ to R₈′ in the chemical formula is an alkoxy group and remainder of the R₁′ to R₈′ is a hydrogen atom, or a mixture thereof.

With this configuration, the cell voltage and electrolyte viscosity can be adjusted by adjusting the mixing ratio of anthraquinones with different properties.

1. An anthraquinone-based active material for a redox flow battery, comprising a first compound represented by the following chemical formula: 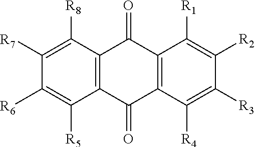
where at least one of the R₁ to R₈ is a hydroxy group, and at least one of the R₁ to R₈ is an alkoxy group.

2. The anthraquinone-based active material according to claim 1, wherein only one of the R₁ to R₈ is a hydroxy group.

3. The anthraquinone-based active material according to claim 2, wherein only one of the R₁ to R₈ is an alkoxy group.

4. The anthraquinone-based active material according to claim 1, wherein only two of the R₁ to R₈ are hydroxy groups.

5. The anthraquinone-based active material according to claim 4, wherein only two of the R₁ to R₈ are alkoxy groups.

6. The anthraquinone-based active material according to claim 5, wherein two of the R₂, R₃, R₆, and R₇ are hydroxy groups and remaining two of the R₂, R₃, R₆, and R₇ are alkoxy groups.

7. The anthraquinone-based active material according to claim 1, wherein the R₂ is a hydroxy group, and the R₆ is an alkoxy group.

8. The anthraquinone-based active material according to claim 1, wherein the alkoxy group is O(CH₂)ₙCOOH, where n is a natural number from 1 to 6.

9. The anthraquinone-based active material according to claim 1, wherein when a molecule of the first compound is inverted with respect to center of a central six-membered ring of anthraquinone skeleton, a combination of R₁ to R₈ has a different structure from that before the inversion.

10. The anthraquinone-based active material according to claim 1, wherein the first compound does not contain a sulfo group.

11. The anthraquinone-based active material according to claim 1, wherein the first compound contains a sulfo group, and wherein the number of sulfo groups is less than the number of hydroxy groups.

12. The anthraquinone-based active material according to claim 1, further comprising a second compound represented by the following chemical formula: 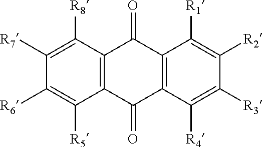
wherein the second compound is a compound in which at least one of the R₁′ to R₈′ is a hydroxy group and remainder of the R₁′ to R₈′ is a hydrogen atom, or a compound in which at least one of the R₁′ to R₈′ in the chemical formula is an alkoxy group and remainder of the R₁′ to R₈′ is a hydrogen atom, or a mixture thereof.
